(19) United States
(12) Patent Application Publication
(10) Pub. No.: US 2011/0008433 A1
(43) Pub. Date: Jan. 13, 2011

(54) Herbal Composition for Osteoarthritis

(76) Inventor: Su-ying LIU, Belmont, CA (US)

Correspondence Address: SINORICA, LLC, 2275 Research Blvd., Suite 500, ROCKVILLE, MD, 20850, US

(21) Appl. No.: 12/501,523

(22) Filed: Jul. 13, 2009

(51) Int. Cl. A61K 9/28; A61K 36/00

(52) U.S. Cl. 424/474; 424/725

(57) ABSTRACT

A novel herbal composition comprising of Lactuca sativa L., Aquilariae resinatum Lignum, Oryza sativa L., Boswellia carterii Birdw, Rhizoma atractylodis macrocephalae and Semen cuscutae is useful in treating osteoarthritis.

### BACKGROUND OF THE INVENTION

1. Technical Field

The present invention relates to a composition for the treatment of osteoarthritis. More particularly, it relates to a composition comprising Chinese herbs.

2. Description of Related Art

Osteoarthritis (OA), the joint degeneration disease, affects more people than any other joint diseases due to the pain and dysfunction it causes.

According to CDC, OA in the USA affects 13.9% of adults aged 25 and older and 33.6% (12.4 million) of those 65 and over. The costs are estimated to be $7.9 billion for knee and hip replacements in 1997. Total annual disease costs US$5700 (year 2000) and job-related OA costs US$3.4 to $13.2 billion per year (www.cdc.gov/arthritis/osteoarthritis.htm).

In western countries, 7-18% of older people have symptomatic osteoarthritis of the knee. A population-based study in China also found that symptomatic osteoarthritis of the knee occurs in 15% of women and 6% of men aged 60 and older (BMC Health Serv Res. 2008; 8: 24).

Disease in weight bearing joints (hip, knee) has greater clinical impact. OA of the knee is 1 of 5 leading causes of disability among non-institutionalized adults. It has tremendous impact on the quality of life of sufferers. About 35˜50% of OA may be genetically determined. Other risk factors include excess body mass, joint injury, excessive mechanical stress, mal-alignment, female gender, and ageing (Int. J. Osteoarchaeology, 2007, 17: 437-450). The intertwined abnormalities of cartilage, bone and synovium lead to joint degeneration.

Cartilage is composed of water, collagen, and proteoglycans. In healthy cartilage, continual internal remodeling occurs as the chondrocytes replace macromolecules lost through degradation. This process becomes disrupted in osteoarthritis. An imbalance of anabolic and catabolic activities of chondrocyte leads to increased degenerative changes and an abnormal repair response. In early OA, there is increased synthetic activity as an attempt to regenerate the matrix (type II, IX, XI, IV collagen; aggrecan) in response to the ongoing enzymatic and biomechanical degradation. Eventually, these reparative processes fail resulting in the destruction of cartilage. The abnormal repair process leads to the formation of osteophytes and subchondral cysts as the disease progresses. Compromised ability of chondrocytes to maintain and restore cartilage tissue damage caused by articular surface mechanical stress has an important role in the development of joint degeneration (J. Cell. Physiol. 2007, 213: 626-634; Osteoarthritis and Cartilage. 2008, 16: S1-S3).

OA is not considered a classical inflammatory arthritis. Inflammation is usually mild. However, it is associated with episodic synovitis with symptoms of inflammation, including pain, swelling and stiffness, and also inflammatory mediator productions of leukotriene (by 5-lipoxygenase) and prostaglandin (by cyclooxygenase). These mediators further up-regulate IL-1β and TNF-α expressions that induce the expression of catabolic enzymes-matrix metalloproteinases (MMPs) and aggrecanases (J. Rheumatol. 29(3): 546-553).

It is well recognized that age is a primary risk factor for the development of OA, and is associated with changes in the matrix composition and a decrease in chondrocyte function and responsiveness to stimuli. Ageing is also associated with the accumulation of advanced glycation end products (AGEs) that is resulted from non-enzymatic oxidation in articular cartilage. Chondrocytes express receptor for advanced glycation end products (RAGE), and its level is correlated with age and the presence of OA. Chondrocyte RAGE signaling increases MMP-1, -3, and -13 productions. These are the MMPs evidently involved in OA (Arthritis Rheum. 2005, 52(8): 2376-2385; Ann Rheum Dis 2005, 64: 891-898; FEBS Lett. 2007, 581(9): 1928-1932).

Dynamic compression and shear force regulate the anabolic and catabolic activities of cartilage. Low load stimulates anabolic activities (aggrecan and type II collagen synthesis) and high load stimulates catabolic activities (MMPs and aggrecanases) in general (J. Biol. Chem. 2006, 281(34): 34095-34103). High shear stress, such as mal-alignment of the joint, induces COX II expression, depletion of anti-oxidants and chondrocyte apoptosis (Proc. Nat. Acad. Sci. 2005, 102(39): 14010-14015; J. Biol. Chem., 2003; 278(31): 28388-28394). Shear stress induces expression of nitric oxide synthetase with increase in nitric oxide production. Increase of nitric oxide has been found in OA synovial fluid, and increase in nitro-adduct (nitrotyrosine) is found in OA cartilage. This oxidative attack is one of the factors causing down-regulation of chondrocyte function, senescence, and cartilage degeneration (Arthritis Res. Ther. 2005, 7: R380-R391).

Oxidation stress exerted by reactive oxygen species (ROS), including nitric oxide (NO), O²⁻, ⁻HO, and H₂O₂, is capable of inducing senescence and apoptotic cell death in chondrocytes. Oxidative stress contributes to OA progression during ageing process. Chondrocytes from older donors are more susceptible to oxidative damages. Chondrocytes in adult articular cartilage do not divide to replace damaged or dead cells. Oxidative stress and accumulation of oxidative damage may be particularly harmful in low turnover tissue like cartilage. A study has shown 22.3% cell death in chondrocyte of OA cartilage (Arthritis Rheum. 1998, 41(9): 1632-1638; Arthritis Rheum. 2003, 48(12): 3419-3430). Chondrocyte senescence caused by ROS and/or advancing age contributes to cartilage degeneration by decreasing the ability of chondrocytes to maintain and repair the articular cartilage tissue. Human OA chondrocytes also demonstrate enhanced lipoxidative activity in lesions (Arthritis Rheum. 2005; 52(9): 2799-2807).

Currently available treatments of OA include: rest, exercise, lose weight, heat and cold pain management, physical therapy, pain killer, NSAID, cortisone, visco-supplementation, and surgery (insertion of soft tissue grafts, penetrating subchondral bone, debridement, realignment, bone fusion, joint replacement) (Advanced Drug Delivery Reviews. 2006; 58 (2): 150-167). Some food supplements, for example, glucosamine, chondroitin sulfate, have not been clinically proven efficacious clearly. There is no disease modifying drug available currently.

### SUMMARY OF THE INVENTION

To remedy the aforementioned shortcomings, the present invention discloses a herbal composition for the treatment of osteoarthritis comprising Lactuca sativa L., Aquilariae resinatum Lignum, Oryza sativa L., Boswellia carterii Birdw, Rhizoma atractylodis macrocephalae and Semen cuscutae at the weight by weight ratio of 5-20:0.2-1:1-5:0.2-1:0.1-1:0.2-1, preferably at the ratio of 5:0.5:4:0.5:0.25:0.5 in the range of 3 to 12 grams of raw material per day to a person in need. A whole plant or a certain part of the herbal plants may be used for the preparation of the pharmaceutical composition. Preferably, resin-containing trunks of the Aquilariae resinatum Lignum, seeds of the Oryza sativa L, seeds of the Lactuca sativa L, and resins of the Boswellia carterii Birdw are used according to the invention.

The present invention also provides a herbal composition for the treatment of osteoarthritis comprising Lactuca sativa L., Aquilariae resinatum Lignum., Oryza sativa L., Boswellia carterii Birdw, Rhizoma atractylodis macrocephalae and Semen cuscutae at the weight by weight ratio of 5-20:0.2-1:1-5:0.2-1:0.1-1:0.2-1 and formulated with a pharmaceutically acceptable carrier, excipient or diluent.

The preferable pharmaceutically acceptable diluents include but are not limited to sorbitol, mannitol, starch, lactose, cellulose in powder form or microcrystalline form, dicalcium phosphate, tricalcium phosphates, sugar and the like. Other pharmaceutically acceptable carriers and excipients include but are not limited to binders, disintegrants, lubricants, glidants, solubility or wet enhancers, complex forming agents, release controlling agents, film formers, plasticizers, colorants, flavoring agents, sweeteners, viscosity enhancers, preservatives, antioxidants and the like.

The pharmaceutical composition may be prepared in a form of a tablet, soft gel, capsule, granule, powder, liquid, solution, cream, lotion, spray, implant, or transdermal patch for various administration routes.

Hence, a primary object of the present invention is to provide an herbal composition for the treatment of osteoarthritis.

The present invention also demonstrates that the herbal composition can target multifactorial etiology and pathology of OA including mechanical stress (NO free radical attack), oxidative stress (ROS, glycation end product, lipid peroxidation), age-related losing vitality and cell numbers of chondrocyte (imbalance in anabolic and catabolic activities, senescence, apoptosis), wear and tear of cartilage (insufficient replenishment, less collagen and glycosaminoglycan synthesis; MMPs and aggrecanase activation), synovitis releasing inflammatory mediators (prostaglandins via cyclooxygenase-II, leukotriene via 5-lipoxygenase) leading symptomatic swelling, pain, and stiffness; disability and loss of locomotor function, and cartilage degeneration and loss in osteoarthritis.

As demonstrated in Experiment 1, the herbal composition SO101C of Example 1 stimulated chondrocyte proliferation mildly.

As demonstrated in Experiment 2, the herbal composition SO101C of Example 1 increased the depositions of collagen and GAGs in chondrocytes significantly at 0.03 mg/ml, and at this concentration, it inhibited alkaline phosphatase activity in chondrocytes. In addition, the herbal composition SO101C of Example 1 also increased the depositions of collagen and GAGs in chondrocytes significantly at 0.02 mg/ml, and had protection effects against the effect of peroxide on collagen and GAG deposition in chondrocytes in the presence of 0.2 mM H₂O₂.

As demonstrated in Experiment 3, the herbal composition SO101C of Example 1 increased the depositions of collagen and GAGs in bone marrow mesenchymal stem cells significantly at 0.01 and 0.03 mg/ml, and inhibited alkaline phosphatase at 0.03 mg/ml. Bone marrow mesenchymal stem cells are condensed by the herbal composition SO101C of Example 1 at 0.03 mg/ml.

As demonstrated in Experiment 4, IC50 of Cyclooxygenase II for the herbal composition SO101C of Example 1 was between 10 to 100 μg/ml.

As demonstrated in Experiment 5, IC50 of 5-Lipooxygenase for the herbal composition SO101C of Example 1 was between 10 and 100 μg/ml.

As demonstrated in Experiment 6, the herbal composition SO101C of Example 1 demonstrated a mild 20% inhibition of MMP-3 at 100 μg/ml.

As demonstrated in Experiment 7, the herbal composition SO101C of Example 1 demonstrated 21% inhibition of MMP-13 at 100 μg/ml.

As demonstrated in Experiment 8, the herbal composition SO101C of Example 1 inhibited latent and active form MMP-2 secreted by chondrocytes mildly.

As demonstrated in Experiment 9, the herbal composition SO101C of Example 1 inhibited MMP-2 activity in cell free system dose dependently.

As demonstrated in Experiment 10, the herbal composition SO101C of Example 1 demonstrated 48% SOD mimetic activity at 100 μg/ml.

As demonstrated in Experiment 11, the herbal composition SO101C of Example 1 dose dependently captured DPPH free radical as the positive control, vitamin C and E.

As demonstrated in Experiment 12, the herbal composition SO101C of Example 1 at 0.1 mg/ml contained 3.34 mM gallic acid equivalent phenolics which is well documented for its anti-oxidation activity.

As demonstrated in Experiment 13, the herbal composition SO101C of Example 1 dose dependently inhibited lipid peroxidation.

As demonstrated in Experiment 14, the herbal composition SO101C of Example 1 stimulated the expression of most of the cartilage metabolism related genes in chondrocytes, including aggrecan, aggrecanase, type II collagen, TGF-P, BMP-2, and alkaline phosphatase. SO101C is also demonstrated to slightly inhibit MMP-2 expression. Compared with glucosamine, SO101C stimulated TGF-β expressions more. The present invention also stimulated the expressions of aggrecan, aggrecanase, MMP-2 and BMP-2 in bone marrow mesenchymal stem cells, and to a less extends of Col I and Col II expressions.

As demonstrated in Experiment 15, the herbal composition SO101C of Example 1 inhibited both acute phase and inflammatory phase of formalin induced pain.

As demonstrated in Experiment 16, after 7 days of oral administration at 300 mg/Kg of the herbal composition SO101C of Example 1 to the mice, the locomotor activity of the mice increased significantly. At 100 mg/Kg, it did not show increase in locomotor activity.

As demonstrated in Experiment 17, there were significant decreases by the herbal composition SO101C of Example 1 in total score summation of pain, stiffness and difficulty in mobility in human. The decrease in lower back pain was most striking at 67.3%, while the pain in hip and knee decrease 65.8%. The stiffness decreased 63.8%. The difficulty in daily living mobility decreased 62.3%. All these parameters specific for assessment of arthritis decreased about 63.8% after three months of intake of the composition of the present invention without apparent side effects. The four extremities had more energy and the locomotor activity of the muscular skeletal system improved significantly.

### BRIEF DESCRIPTIONS OF THE FIGURES

FIG. 1 demonstrates DPPH free radical scavenging activity. A: SO101C (mg/ml), VitC: vitamin C (mM), VitE: vitamin E (mM).

FIG. 2 demonstrates inhibition of lipid peroxidation. A: SO101C (mg/ml), VitC: vitamin C (mM), VitE: vitamin E (mM).

FIG. 3 demonstrates inhibition of formalin-induced pain. A: SO101C.

FIG. 4 demonstrates enhancement of loaded forced swimming. A: SO101C.

### DETAILED DESCRIPTION OF THE PREFERRED EMBODIMENT

The present invention is a herbal composition for the treatment of osteoarthritis comprising Lactuca sativa L., Aquilariae resinatum Lignum., Oryza sativa L., Boswellia carterii Birdw, Rhizoma atractylodis macrocephalae and Semen cuscutae at the weight by weight ratio of 5-20:0.2-1:1-5:0.2-1:0.1-1:0.2-1, preferably at the ratio of 5:0.5:4:0.5:0.25:0.5 in the range of 3 to 12 grams raw material per day to a person in need. A whole plant or a certain part of the herbal plants may be used for the preparation of the pharmaceutical composition. Preferably, resin-containing trunks of the Aquilariae resinatum Lignum, seeds of the Oryza sativa L, seeds of the Lactuca sativa L, and resins of the Boswellia carterii Birdw are used according to the invention.

The herbal composition is formulated with pharmaceutically acceptable excipients, carriers, or diluents into lozenge, tablet, film coated tablet, capsule, soft capsule, granule, powder, pill, solution, emulsion, injection solution, injection, ointment, cream, spray, inhalant, soft gel, liquid, lotion, implant, or transdermal patch for various administration routes.

### EXAMPLE 1

### Preparation of the Herbal Composition SO101C Powder

150 g of Lignum aquilariae resinatum, 600 g of Fructus oryzae, 150 g of Resina boswelliae carterii, 1500 g of Semen lactucae sativae, 75 g of Rhizoma atractylodis macrocephalae and 150 g of Semen cuscutae were soaked in deionized water and heated to 100.degree. C. for 2.5 hours twice. The extracted fluid was collected and pooled, passed through 100 mesh sieve, concentrated and spray dried into powder (designated as SO101C).

### Experiment 1

### Stimulation of Chondrocyte Proliferation of the Present Invention

### (1-a) Preparation of Human Articular Chondrocyte

Human articular cartilage was obtained from patients who received total joint replacement with consent. The cartilage was digested with 1 mg/ml of Class 2 and Class 4 bacterial collagenase (Worthington Chemical, Freehold, N.J.) in DMEM containing 10% fetal calf serum (FCS) and 25 μg/ml Gentamicin (C-DMEM, Gibco, Grand Island, N.Y.) (C-DMEM) at 37° C. overnight. The primary cells were plated at high density in 10 mm culture dish (Nunc, Roskilde, Danmark) in C-DMEM and cultured under humidified atmosphere containing 5% CO₂ at 37° C.

### (1-b) Chondrocyte Proliferation Assay

1×10⁴ cells in 100 μl of C-DMEM were plated into each well of 96 multiwell dishes (Nunc, Roskilde, Danmark). After 48 hours, the media were replaced with fresh C-DMEM containing designated concentrations of herbal composition of the present invention, SO101C. 72 hours later, the media were aspirated, washed once with PBS, 50 μl of MTT (0.5 mg/ml in PBS) was added, and cells were incubated at 37° C. in humidified atmosphere containing 5% CO₂ for 4 hours. At the end of the incubation, solutions were aspirated, and the reduced formazan was dissolved in 100 μl of isopropanol containing 0.04 N HCl. The absorbance at 570 nm was measured by an ELISA reader (Molecular Device).

TABLE 1
Stimulation of chondrocyte proliferation

| | | SO101C |
|---|---|---|
| Concentration (mg/ml) | Mean OD | % Change of control |
| blank | 0.040 | |
| 0 | 0.174 | |
| 0.1 | 0.239 | 36 |
| 0.05 | 0.238 | 35 |
| 0.025 | 0.212 | 17 |
| 0.0125 | 0.211 | 16 |
| 0.0062 | 0.210 | 16 |

As shown in Table 1, the present invention stimulated chondrocyte proliferation mildly. The proliferated cell number was in the range for replenishing the cell loss during ageing.

### Experiment 2

### Stimulation of ALP, Collagen and GAGs Synthesis in Chondrocytes by Herbal Preparation in Micromass Culture

### (2-a) Micromass Culture System

Ten μl of 1×10⁷ cells/ml in C-DMEM was seeded into the center of each well of 48 multiwell culture dish (Nunc, Roskilde, Danmark). Two hours later, 250 μl of F12/DMEM containing 5% FCS, 6.25 μg/ml bovine insulin, 25 μg/ml ascorbic acid and 100 nM Dexamethasone (induction medium) with various concentrations of herbal composition or glucosamine (Sigma) was added. The media were changed twice a week for two weeks. At the end of the incubation, the media were changed with fresh DMEM. Twenty-four hours later, the media were collected for MMP zymography study and cells were fixed and stained for alkaline phosphatase (ALP), collagen or glycosaminoglycan using Naphthol AS-MX phosphate/Fast red, Sirius red, or Safranin O (Sigma), respectively.

### (2-b) Stimulation of ALP, Collagen and GAGs Synthesis in Chondrocytes by the Herbal Composition in Micromass Culture without H₂O₂

TABLE 2
Stimulation of ALP, collagen and GAGs synthesis in chondrocytes
by herbal composition in micromass culture

| | Conc. | | | |
|---|---|---|---|---|
| | SO101C | | Glucosamine | Control |
| Item | 0.01 mg/ml | 0.03 mg/ml | 1 mM | 0 |
| ALP | ++ | + | ++ | ++ |
| Collagen | +++ | +++ | + | + |
| GAGs | + | ++ | + | + |

As shown in Table 2, the present invention increased the depositions of collagen and GAGs significantly at 0.03 mg/ml, and at this concentration, it inhibited alkaline phosphatase activity. Under the same experimental conditions, glucosamine at 1 mM did not show significant effects.

### (2-c) Stimulation of ALP, Collagen and GAGs Synthesis in Chondrocytes by Herbal Preparation in Micromass culture with H₂O₂

TABLE 3
Stimulation of ALP, collagen and GAGs synthesis in chondrocytes
by herbal composition in micromass culture with 0.2 mM H₂O₂

| Treatment | |
|---|---|
| Without H₂O₂ | With H₂O₂ |

| | SO101C | | SO101C | |
|---|---|---|---|---|
| Item | (0.02 mg/ml) | Control | (0.02 mg/ml) | Control |
| ALP | + | + | ++ | ++ |
| Collagen | ++++ | +++ | ++ | + |
| GAGs | +++ | ++ | ++ | + |

As shown in Table 3, the present invention increased the depositions of collagen and GAGs significantly at 0.02 mg/ml. In the presence of 0.2 mM H₂O₂, the present invention had protection effects against the effect of peroxide on collagen and GAG deposition.

### Experiment 3

### Stimulation of ALP, Collagen and GAGs Synthesis in Bone Marrow Mesenchymal Stem Cells by Herbal Composition in Micromass Culture

### (3-a) Preparation of Human Bone Marrow Mesenchymal Stem Cell

Human bone marrow mesenchymal stem cells were obtained from patients receiving total joint replacement with consent. The cells were cultured in DMEM containing 10% fetal calf serum (FCS) and 25 μg/ml Gentamicin (C-DMEM, Gibco, Grand Island, N.Y.) at 37° C. under humidified atmosphere containing 5% CO₂.

### (3-b) Human Bone Marrow Mesenchymal Stem Cell Proliferation Assay

Ten μl of 1×10⁷ cells/ml in C-DMEM was seeded into the center of each well of 48 multiwell culture dish (Nunc, Roskilde, Danmark). Two hours later, 250 μl of F12/DMEM containing 5% FCS, 6.25 μg/ml bovine insulin, 25 μg/ml ascorbic acid and 100 nM Dexamethasone (induction medium) with various concentrations of herbal composition or glucosamine (Sigma) was added. The media were changed twice a week for two weeks. At the end of the incubation, cells were fixed and stained for alkaline phosphatase (ALP), collagen or glycosaminoglycan using Naphthol AS-MX phosphate/Fast red, Sirius red, or Safranin O (Sigma), respectively.

TABLE 4
Stimulation of ALP, collagen and GAGs synthesis in bone marrow
mesenchymal stem cells by herbal composition in micromass
culture

| | Conc. | | | |
|---|---|---|---|---|
| | SO101C | | Glucosamine | Control |
| Item | 0.01 mg/ml | 0.03 mg/ml* | 1 mM | 0 |
| ALP | ++ | + | ++ | ++ |
| Collagen | ++ | +++ | + | + |
| GAGs | ++ | +++ | + | + |

*Treated with the present invention, the bone marrow mesenchymal stem cells condensed.

As shown in Table 4, the present invention increased the depositions of collagen and GAGs significantly at 0.01 and 0.03 mg/ml, and inhibited alkaline phosphates at 0.03 mg/ml. Bone marrow mesenchymal stem cells are condensed by the present invention at 0.03 mg/ml. This was similar as in limb bud during development. ALP was thought to be involved in bone formation. Abnormal repair of cartilage leading to osteophyte formation was not desirable. SO101C inhibited ALP may have effect on prevention of osteophyte formation. Under the same experimental conditions, glucosamine at 1 mM did not show significant effect.

### Experiment 4

### Inhibition of Cyclooxygenase II (COX II) Activity by the Present Invention

Human recombinant COX II expressed in Sf21 cells (Sigma, C-0858) was used. Herbal composition at 10 or 100 μg/ml or vehicle was preincubated with 0.11 U enzyme, 1 mM reduced glutathione, 500 μM phenol and 1 μM hematin in Tris-HCl, pH 7.7 at 37° C. for 15 minutes. 0.3 μM arachidonic acid as substrate was added and the reaction was terminated by addition of 1 N HCl after 5 minutes. The converted PGE₂ was measured by an Amersham EIA kit following centrifugation.

TABLE 5
Inhibition of COX II by SO101C

| | Concentration | | % Inhibition |
|---|---|---|---|
| SO101C | 10 | μg/ml | 24 |
| SO101C | 100 | μg/ml | 92 |
| Nimesulide | 0.3083 | μg/ml | 50 |

As shown in Table 5, IC50 for SO101C was between 10 to 100 μg/ml. The IC50 for standard COX II inhibitor, Nimesulide, was 1 μM (0.3083 μg/ml).

### Experiment 5

### Inhibition of 5-Lipooxygenase (5-LO) Activity of the Present Invention

Human peripheral blood mononuclear leukocytes (PBML) were isolated by Ficoll-Paque density gradient centrifugation. SO101C at 10 or 100 μg/ml or vehicle was preincubated with PBML (5×10⁶ cells/ml) in HBSS buffer, pH 7.4 at 37° C. for 15 minutes. After incubation with 30 μM A23187 for 15 minutes, the reaction was terminated by addition of 1 N HCl. Following neutralization with NaOH and centrifugation, LTB₄ in the supernatant was measured using an EIA kit.

TABLE 6
Inhibition of 5-LO by SO101C

| | Concentration | | % Inhibition |
|---|---|---|---|
| SO101C | 10 | μg/ml | 36 |
| SO101C | 100 | μg/ml | 95 |
| NDGA | 0.0393 | μg/ml | 50 |

NDGA = nordihydroguaretic acid

As shown in Table 6, IC50 for SO101C was between 10 and 100 μg/ml. The IC50 for standard 5-LO inhibitor, nordihydroguaretic acid, was 0.13 μM (0.0393 μg/ml).

### Experiment 6

### Inhibition of MMP-3 Enzyme Activity of the Present Invention

MMP-3 (stromelysin-1, human recombinant catalytic domain from Calbiochem Cat. 444217) was activated by p-aminophenylmercuric acetate for 60 minutes at 37° C. After preincubation of SO101C or vehicle with the activated enzyme (5 nM) in a reaction mixture containing 50 mM MOPS (pH 7.2), 10 mM CaCl₂ and 10 μM ZnCl₂ at 37° C. for 60 minutes, the reaction was initiated by addition of 4 μM (7-methoxycoumarin-4-yl)acetyl-Pro-Leu-Gly-Leu-N-3-(2,4-dinitrophenyl)-L-2,3-diaminopropinoyl-Ala-Arg-NH₂ and incubated for a further 120 minutes at 37° C. Enzyme activity was determined spectrofluorometrically by measuring the formation of fluorescent (7-methoxycoumarin-4-yl)acetyl-Pro-Leu-Gly.

TABLE 7
MMP-3 activity inhibition by SO101C

| Treatment | Concentration | | Inhibition (%) |
|---|---|---|---|
| TIMP-1 | 0.0076 | μM | 50 |
| TIMP-2* | 0.0077 | μM | 50 |
| SO101C | 100 | μg/ml | 20 |

*Indicates standard reference agent used.

As shown in Table 7, SO101C demonstrated a mild 20% inhibition of MMP-3 at 100 μg/ml.

### Experiment 7

### Inhibition of MMP-13 Enzyme Activity by the Present Invention

MMP-13 proenzyme (human recombinant, expressed in Sf9 cell, Calbiochem Cat. 444248) was activated by p-aminophenylmercuric acetate for 60 minutes at 37° C. After preincubation of SO101C or vehicle with the activated enzyme with the activated enzyme (0.5 nM) in a reaction mixture containing 50 mM MOPS (pH 7.2), 10 mM CaCl₂ and 10 μM ZnCl₂ for 60 minutes at 37° C., the reaction was initiated by addition of 4 μM (7-methoxycoumarin-4-yl)acetyl-Pro-Leu-Gly-Leu-N-3-(2,4-dinitrophenyl)-L-2,3-diaminopropinoyl-Ala-Arg-NH₂ and incubated for a further 120 minutes at 37° C. Enzyme activity was determined spectrofluorometrically by measuring the formation of fluorescent (7-methoxycoumarin-4-yl)acetyl-Pro-Leu-Gly.

TABLE 8
MMP-13 activity inhibition by SO101C

| Treatment | Concentration | | Inhibition (%) |
|---|---|---|---|
| TIMP-1 | 0.0068 | μM | 50 |
| TIMP-2* | 0.0028 | μM | 50 |
| SO101C | 100 | μg/ml | 21 |

*Indicates standard reference agent used.

As shown in Table 8, SO101C demonstrated 21% inhibition of MMP-13 at 100 μg/ml.

### Experiment 8

### Inhibition of MMP-2 Activities of Chondrocyte by the Present Invention in Zymography Assay

The protein concentrations of SO101C (0.01 mg/ml, 2 weeks) or glucosamine (1 mM, 2 weeks) or vehicle treated serum free culture supernatants of chondrocytes were measured by Bio-Rad protein assay solution. Equal amount of protein of each culture medium was mixed with equal volume of 2 fold concentration of non-reducing SDS sample buffer, and incubated at room temperature for two hours for sample to denature. The samples were loaded into each well of a 10% SDS polyacrylamide gel containing 1 mg/ml of bovine gelatin (Sigma), and run at 20 mA in ice bath by a BioRad Mini Protean gel apparatus. After dye front reached the bottom of the gel, the gel was washed three times, 15 minutes each, with 2.5% Triton X-100. Then, the gel was renatured in developing buffer (50 mM Tris-HCl, pH7.6, 10 mM CaCl₂, 50 mM NaCl, 0.05% Brij35) at 37° C. for 24 hours. The gel was stained with 0.1% Coomassie blue R250 (Sigma) in 40% methanol and 10% acetic acid, and destained in 30% methanol and 7% acetic acid. The enzyme digested clear bands were visualized in the blue background. The comparison of the enzymatic activities (clear band width) was analyzed by Image J (NIH).

TABLE 9
MMP-2 activities of chondrocyte treated by SO101C or glucosamine

| | MMP-2 activity (% of control) | |
|---|---|---|
| Treatment | Latent form (72K) | Active form (66K) |
| SO101C (0.02 mg/ml) | 92.9 | 89.5 |
| Glucosamine (1 mM) | 92.5 | 118.2 |

As shown in Table 9, the present invention inhibited latent form and active form MMP-2 secreated by chondrocyte mildly.

### Experiment 9

### Inhibition of MMP-2 Activity in Cell Free System

Serum free culture supernatant of confluent human dermal fibroblast was used as the source of MMP. Equal amount of the supernatant treated with equal volume of 2 fold concentration of SDS sample buffer without reducing agent was subjected to electrophoresis The samples were loaded into each well of a 10% SDS polyacrylamide gel containing 1 mg/ml of bovine gelatin (Sigma), and run at 20 mA in ice bath by a BioRad Mini Protean gel apparatus. After dye front reached the bottom of the gel, the gel was washed three times, 15 minutes each, with 2.5% Triton X-100. Then, the gel was sliced and incubated in developing buffer (50 mM Tris-HCl, pH7.6, 10 mM CaCl₂, 50 mM NaCl, 0.05% Brij35) containing SO101C or EDTA or vehicle at 37° C. for 24 hours. The gel was stained with 0.1% Coomassie blue R250 (Sigma) in 40% methanol and 10% acetic acid, and destained in 30% methanol and 7% acetic acid. The enzyme digested clear bands were visualized in the blue background. The comparison of the enzymatic activities (clear band width) was analyzed by Image J (NIH).

TABLE 10
MMP-2 activities treated by SO101C or EDTA in cell free system

| | |
|---|---|
| Treatment | MMP-2 activity (% of control) |

SO101C

| | |
|---|---|
| 0.1 mg/ml | 79.5 |
| 0.3 mg/ml | 71.5 |
| 1.0 mg/ml | 37.9 |

EDTA

| | |
|---|---|
| 0.1 mM | 46.6 |
| 0.3 mM | 30.5 |

As shown in Table 10, the present invention inhibited MMP-2 activity dose dependently. The non-specific MMP inhibitor, EDTA, also showed dose dependent inhibition in the cell free system.

### Experiment 10

### Inhibition of Free Radical Scavenger, SOD Mimetic Activity by the Present Invention

SO101C or vehicle was incubated with 0.12 mM xanthine, 6 mU xanthine oxidase, 27 μM nitroblue tetrazolium (NBT), 0.11 mM EDTA, 0.005% bovine serum albumin and Na₂CO₃ at pH 10.5 at 25° C. for 20 minutes. Conversion of xanthine to uric acid +O⁻+NBT to formazan was then determined by measurement of absorbance at 595 nm and percent inhibition by superoxide dismutase or test compound was calculated.

TABLE 11
Free radical scavenger, SOD mimetic activity by SO101C

| Treatment | Concentration | | Inhibition (%) |
|---|---|---|---|
| (−)-Epicatechin gallate | 6.1 | μM | 50 |
| Superoxide dismutase (SOD)* | 0.0084 | μM | 50 |
| SO101C | 100 | μg/ml | 48 |

As shown in Table 11, SO101C demonstrated 48% SOD mimetic activity at 100 μg/ml.

### Experiment 11

### 2,2-diphenyl-1-picrylhydrazyl (DPPH) Radical Scavenging Activity of the Present Invention

SO101C, vitamin C or vitamin E at various concentrations were incubated with 0.4 mM 2,2-diphenyl-1-picrylhydrazyl (DPPH, Sigma) in ethanol at room temperature for 30 minutes. Absorbance at 517 nm was measured by a spectrophotometer.

TABLE 12
2,2-diphenyl-1-picrylhydrazyl (DPPH) radical scavenging activity

| | | SO101C (mg/ml) | | | | | Vitamin C (mM) | | | Vitamin E (mM) | | |
|---|---|---|---|---|---|---|---|---|---|---|---|---|
| | Control | 0.01 | 0.03 | 0.1 | 0.3 | 1 | 0.01 | 0.03 | 0.1 | 0.01 | 0.03 | 0.1 |
| Sample 1 | 0.955 | 0.858 | 0.746 | 0.441 | 0.273 | 0.100 | 0.753 | 0.321 | 0.068 | 0.647 | 0.213 | 0.204 |
| Sample 2 | 0.964 | 0.879 | 0.752 | 0.416 | 0.258 | 0.097 | 0.790 | 0.362 | 0.079 | 0.688 | 0.208 | 0.241 |
| Sample 3 | 0.971 | 0.878 | 0.763 | 0.419 | 0.261 | 0.095 | 0.781 | 0.333 | 0.068 | 0.668 | 0.245 | 0.205 |
| Average | 0.963 | 0.872 | 0.754 | 0.425 | 0.264 | 0.097 | 0.775 | 0.339 | 0.072 | 0.668 | 0.222 | 0.217 |
| SD | 0.008 | 0.012 | 0.009 | 0.014 | 0.008 | 0.003 | 0.019 | 0.021 | 0.006 | 0.021 | 0.020 | 0.021 |
| CV % | 0.8 | 1.4 | 1.2 | 3.3 | 3.0 | 3.1 | 2.4 | 6.2 | 8.3 | 3.1 | 9.0 | 9.7 |
| Inhibition (%) | | 9.4 | 21.7 | 55.9 | 72.6 | 89.9 | 19.5 | 64.8 | 92.5 | 30.6 | 76.9 | 77.5 |

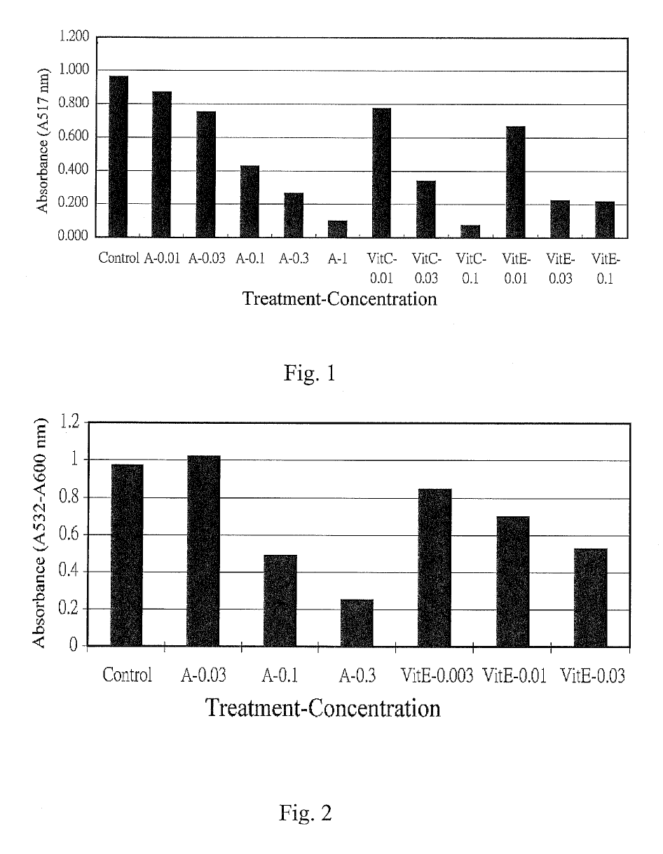

As shown in FIG. 1 which was the figure of Table 12, the present invention dose dependently captured DPPH free radical as the positive control, vitamin C and E did.

### Experiment 12

### Total Phenolics as Gallic Acid (GA.) Equivalent of the Present Invention

Various concentrations of SO101C were incubated with Folin-Ciocalteu's phenol reagent (final concentration: 6.25%) in 3.75% Na₂CO₃ at room temperature for 2 hours in dark using gallic acid (Sigma) as standard. Absorbance at 765 nm was measured by a spectrophotometer.

TABLE 13
Total phenolics of the present invention

| | |
|---|---|
| Concentration | G.A. equivalent |

SO101C

| | |
|---|---|
| 0.01 mg/ml | 0.13 mM |
| 0.03 mg/ml | 1.03 mM |
| 0.10 mg/ml | 3.34 mM |

Vitamin C

| | |
|---|---|
| 0.01 mM | 1.15 mM |
| 0.03 mM | 2.34 mM |
| 0.10 mM | 7.20 mM |

Vitamin E

| | |
|---|---|
| 0.01 mM | 1.05 mM |
| 0.03 mM | 2.36 mM |
| 0.10 mM | 3.71 mM |

As shown in Table 13, the present invention at 0.1 mg/ml contained 3.34 mM gallic acid equivalent phenolics which is well documented for its anti-oxidation activity.

### Experiment 13

### Anti-Lipid Peroxidation Activity of the Present Invention

Mouse brain KCl extract was used as the source of lipid. Brain extract was incubated with 1 mM FeSO₄ and 0.1 mM vitamin C at 37° C. for 90 minutes. The malondialdehyde formed by peroxyl radical was reacted with thiobarbituric acid to form an adduct that absorbed at 532 nm after centrifugation to discard TCA precipitation.

TABLE 14
Inhibition of lipid peroxidation by the present invention

| | |
|---|---|
| Concentration | OD (A₅₃₂-A₆₀₀) |

SO101C

| | | |
|---|---|---|
| 0 | mg/ml | 0.973 |
| 0.03 | mg/ml | 1.023 |
| 0.1 | mg/ml | 0.486 |
| 0.3 | mg/ml | 0.250 |

Vitamin E

| | | |
|---|---|---|
| 0 | mM | 0.973 |
| 0.003 | mM | 0.844 |
| 0.01 | mM | 0.697 |
| 0.03 | mM | 0.526 |

As shown in FIG. 2 which was the figure of Table 14, SO101C and vitamin E dose dependently inhibited lipid peroxidation.

### Experiment 14

### Effect of the Present Invention on Gene Expressions in Chondrocytes or Bone Marrow Mesenchymal Stem Cells

Confluent monolayer chondrocyte cells or micromass bone marrow mesenchymal stem cells were treated with SO101C (0.01 mg/ml) or glucosamine (1 mM) or vehicle in F12/DMEM containing 5% FCS, 6.25 μg/ml bovine insulin, 25 μg/ml ascorbic acid and 100 nM Dexamethasone for 2 weeks with medium changed twice a week. At the end of each treatment, the cells were washed with PBS, and RNA was extracted by Tri-Reagent (Sigma). 800 ng of total RNA was reverse transcribed by Omniscript reverse transcriptase (Qiagen, Valencia, Calif.) in buffer containing 1 uM random primer, 0.5 mM each of dNTP, and 10 units of ribonuclease inhibitor. Reverse transcription was carried out at 37° C. for one hour by a programmable thermal controller (PTC-100, MJ Research, Inc., Watertown, Mass.). Reaction was terminated by raising the temperature to 93° C. for five minutes.

Polymerase chain reaction (PCR) amplification was performed with primer pairs as listed in Table 15. PCR was performed in a thermal controller (PTC-100, MJ Research, Inc., Watertown, Mass.) with master mix (HotStarTaq DNA polymerase, Qiagen) at initial denaturation at 95° C. for 15 minutes, followed by 25 cycles of 94° C. for 1 minute, 58° C. for 40 seconds, and 72° C. for 40 seconds. The final cycle extended 72° C. for 10 minutes. The PCR products accompanied by 100 bp ET marker were separated by 1.2% agarose gel, stained with ethidium bromide and visualized under UV light. A Polaroid picture was taken by Foto/Phoresis UV documentation system (Fotodyne Inc.). Beta-actin expression under the same experimental conditions was used as a reference to normalize the expressions. The comparison of bend width of PCR was analyzed by Image J (NIH) after normalized by the expression of β-Actin.

TABLE 15
Nucleotide sequence of primers used in PCR

| | |
|---|---|
| Gene | Primer sequence from 5′ to 3′ |

| | |
|---|---|
| Col I | |
| Forward | CGT GGT GAC AAG GGT GAG AC |
| Reverse | TAG GTG ATG TTC TGG GAG GC |
| Col II | |
| Forward | CTG GCT CCC AAC ACT GCC AAC GTC |
| Reverse | TCC TTT GGG TTT GCA ACG GAT TGT |
| Aggrecan | |
| Forward | CAC TGT TAC CGC CAC TTC CC |
| Reverse | GAG ATC GTT CCT CTC GCC CT |
| Aggrecanase | |
| Forward | AGA TCC AGG AAA GGA GGG CT |
| Reverse | AAA GCT CTC TGT AGC CT |
| MMP-2 | |
| Forward | GAT GCC TGG AAT GCC ATC CCC GAT |
| Reverse | AGG CTT GGT GGG ATT GGA GGG GGA |
| BMP-2 | |
| Forward | CAG AGA CCC ACC CCC AGC A |
| Reverse | CTG TTT GTG TTT GGC TTG AC |
| TGF-β | |
| Forward | TGA ACC GGC CTT TCC TGC TTC TCA TG |
| Reverse | GCG GAA GTC AAT GTA CAG CTG CCG C |
| MMP-9 | |
| Forward | GAG GTT CGA CGT GAA GGC GCA GAT G |
| Reverse | CAT AGG TCA CGT AGC CCA CTT GGT C |
| ALP | |
| Forward | TGG AGC TTC AGA AGC TCA ACA CCA |
| Reverse | ATC TCG TTG TCT GAG TAC CAG TCC |
| β-Actin | |
| Forward | CAG GTC ATC ACY ATY GGC AAT GAG C |
| Reverse | CGG ATG TCM ACG TCA CAC TTC ATG A |

TABLE 16
Gene expression of chondrocyte treated by SO101C or glucosamine

Gene expression
(% of control)
Treatment

| Gene | SO101C | glucosamine |
|---|---|---|
| Aggrecan | 132.9 | 133.7 |
| Aggrecanase | 128.7 | 130.9 |
| Col I | 83.3 | 82.7 |
| Col II | 135.8 | 162.4 |
| MMP-2 | 70.7 | 83.9 |
| ALP | 149.3 | 180.0 |
| BMP-2 | 174.1 | 173.4 |
| TGF-β | 139.0 | 120.9 |

As shown in Table 16 (effect of SO101C on gene expression of human chondrocytes normalized by the expression of β-Actin and analyzed by Image J), SO101C and glucosamine stimulated the expression of most of the cartilage metabolism related genes, including aggrecan, aggrecanase, type II collagen, TGF-β, BMP-2, and alkaline phosphatase. There was some inhibition in expression on type I collagen by both SO101C and glucosamine. SO101C and glucosamine inhibited MMP-2 expression. Compared with glucosamine, SO101C stimulated TGF-β expressions more, however, glucosamine stimulated type II collagen and alkaline phosphatase expressions more.

TABLE 17
Gene expression of bone marrow mesenchymal stem cells treated by
SO101C normalized by expression of β-Actin

| Gene | Gene expression (% of control) |
|---|---|
| Aggrecan | 132.6 |
| Aggrecanase | 152.2 |
| Col I | 108.6 |
| Col II | 107.4 |
| MMP-2 | 127.8 |
| BMP-2 | 136.1 |

As shown in Table 17, SO101C slightly increased the expressions of Col I and Col II in micromass culture of bone marrow mesencymal stem cells. Micromass culture treated by SO101C induced the expression of aggrecan, aggrecanase, MMP-2, and BMP-2. There was no detectable expression of MMP-9 in bone marrow mesenchymal stem cells in control or SO101C treatment cells under this experimental condition (data not shown).

### Experiment 15

### Effect of the Present Invention on the Flinching Numbers of Formalin Induced Pain in Mice

Male ICR mice at the age of 4 weeks (BW˜20 g) were given SO101C at 30 mg/Kg or vehicle control via gavage for two weeks. Twenty μl of 5% formalin in saline was injected into the right hind paw subcutaneously. The flinching of the injected paw was counted for 30 minutes in a 5-minute block.

TABLE 18
Effect of SO101C on the flinching numbers of formalin induced pain

Flinching number

| Time (minutes) | Control | SO101C (30 mg/Kg) |
|---|---|---|
| 0-5 | 218 | 133 |
| 5-10 | 26 | 2 |
| 10-15 | 0 | 3 |
| 15-20 | 37 | 21 |
| 20-25 | 65 | 10 |
| 25-30 | 45 | 25 |

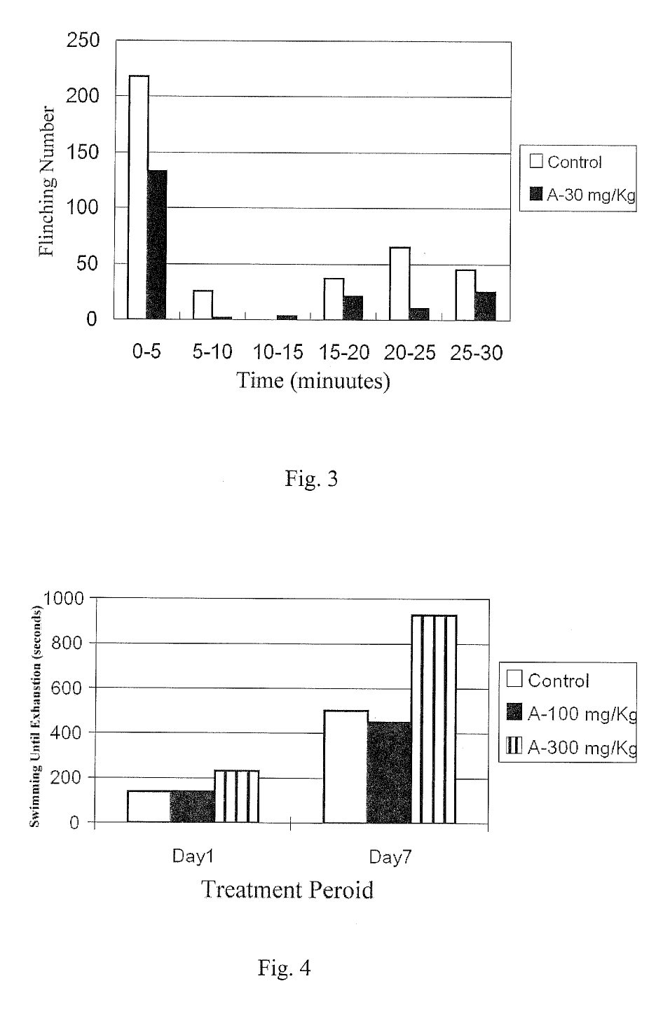

As shown in FIG. 3 which was the figure of Table 18, the present invention inhibited both acute phase and inflammatory phase of formalin induced pain.

### Experiment 16

### Effect of the Present Invention on Loaded Forced Swimming in Mice

Male ICR mice (6 weeks old) were administered SO101C at 0, 100, or 300 mg/Kg by oral gavage. On day 1 and 7 of treatments, the mice were loaded with 10% of their body weights, and forced to swim in a water pool kept at 25° C. The duration of time of swimming until exhaustion (both nostrils were submerged underneath of the water surface for 7 seconds) was recorded.

TABLE 19
Effect of SO101C on loaded forced swimming until exhaustion in mice

| Treatment | Day 1 | Day 7 |
|---|---|---|
| Control | 139 sec | 499 sec |
| 100 mg/Kg | 139 sec | 448 sec |
| 300 mg/Kg | 234 sec | 927 sec |

As shown in FIG. 4 which is the figure of Table 19, after 7 days of oral administration at 300 mg/Kg of the present invention to the mice, the locomotor activity of the mice increased significantly. At 100 mg/Kg, it did not show increase in locomotor activity.

### Experiment 17

### Human Use Experiences

The assessment questionnaire was designed according to pain and movement of arthritis. 15 females with apparent arthritis symptoms were instructed to fill in the questionnaire at baseline and 12 weeks after intake of the extract powder of the present invention at 900 mg/day, BID, by scoring the degree of pain, stiffness and movement as None: 0, Slight: 1, Mild: 2, Moderate: 3, Severe: 4.

TABLE 20
Total score summation changes of 15 participants in pain,
stiffness and mobility by SO101C

| | Baseline | 12 Weeks | % Change |
|---|---|---|---|
| Lower back pain | 26.0 | 8.5 | −67.3 |
| Hip or knee joint pain while: | | | |
| Walking or flat ground | 10.0 | 1.5 | −85.0 |
| Going up and down the stairs | 15.0 | 3.0 | −80.0 |
| Lying on bed | 10.5 | 4.0 | −61.9 |
| Sitting | 11.0 | 7.0 | −36.4 |
| Standing | 13.5 | 5.0 | −63.0 |
| Subtotal: | 60.0 | 20.5 | −65.8 |
| Stiffness: | | | |
| Wake up in the morning | 27.0 | 9.0 | −66.7 |
| After sitting, lying or resting | 25.5 | 10.0 | −60.8 |
| Subtotal: | 52.5 | 19.0 | −63.8 |
| Difficulty in mobility: | | | |
| Up and down stairs | 11.0 | 3.0 | −72.7 |
| Standing up and sitting down | 19.5 | 10.0 | −48.7 |
| Bending | 15.0 | 7.0 | −53.3 |
| Flat ground walking | 13.0 | 4.0 | −69.2 |
| In and out of car | 12.5 | 5.0 | −60.0 |
| Lying down and getting out of | 7.5 | 1.0 | −86.7 |
| bed | | | |
| Putting on and taking off socks | 12.0 | 4.0 | −66.7 |
| In and out of bath tub | 11.0 | 4.0 | −63.6 |
| Shopping | 3.0 | 2.0 | −33.3 |
| Heavy housekeeping work | 17.5 | 7.0 | −60.0 |
| Light housekeeping work | 10.5 | 3.0 | −71.4 |
| Subtotal: | 132.5 | 50.0 | −62.3 |
| Total | 271.0 | 98.0 | −63.8 |

As shown in Table 20, there were significant decreases in total score summation of pain, stiffness and difficulty in mobility. The decrease in lower back pain was most striking at 67.3%, while the pain in hip and knee decrease 65.8%. The stiffness decreased 63.8%. The difficulty in daily living mobility decreased 62.3%. All these parameters specific for assessment of arthritis decreased about 63.8% after three months of intake of the composition of the present invention without apparent side effects. The four extremities had more energy and the locomotor activity of the muscular skeletal system improved significantly.

The present invention has been described by reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed:

1. An herbal composition comprising Lactuca sativa L., Aquilariae resinatum Lignum., Oryza sativa L., Boswellia carterii Birdw, Rhizoma atractylodis macrocephalae and Semen cuscutae at a weight by weight ratio of 5-20:0.2-1:1-5:0.2-1:0.1-1:0.2-1, wherein said herbal composition is for the treatment of osteoarthritis.

2. The herbal composition of claim 1, wherein said ratio is preferable at the weight by weight ratio of 5:0.5:4:0.5:0.25:0.5.

3. The herbal composition of claim 1, having an effectively therapeutic weight that is in the range of 3 to 12 grams of raw material per day.

4. The herbal composition of claim 1, wherein Aquilariae resinatum Lignum comprises resin-containing trunks of the Aquilariae resinatum Lignum.

5. The herbal composition of claim 1, wherein Oryza sativa L comprises seeds of the Oryza sativa L.

6. The herbal composition of claim 1, wherein Lactuca sativa L comprises seeds of the Lactuca sativa L.

7. The herbal composition of claim 1, wherein the Boswellia carterii Birdw comprises resins of the Boswellia carterii Birdw.

8. The herbal composition of claim 1 comprising an ability to stimulate the proliferation of chondrocyte.

9. The herbal composition of claim 1 comprising an ability to stimulate the deposition of collagen and glycosaminoglycan by chondrocyte.

10. The herbal composition of claim 1 comprising an ability to stimulate human bone marrow mesenchymal stem cell to differentiate into a chondrocyte lineage.

11. The herbal composition of claim 1 comprising an ability to stimulate cartilage metabolic related genes in chondrocytes.

12. The herbal composition of claim 1 comprising an ability to stimulate cartilage metabolic related genes in bone marrow mesenchymal stem cells

13. The herbal composition of claim 1 having an anti-MMP-2 activity.

14. The herbal composition of claim 1 having an anti-MMP-3 activity.

15. The herbal composition of claim 1 having an anti-MMP-13 activity.

16. The herbal composition of claim 1 having an anti-oxidation activity.

17. The herbal composition of claim 1 having an anti-inflammatory activity.

18. The herbal composition of claim 1 having anti-pain activity.

19. The herbal composition of claim 1 having a locomotor enhancement activity.

20. The herbal composition of claim 1 is formulated with pharmaceutically acceptable excipients, carriers, or diluents into lozenge, tablet, film coated tablet, capsule, soft capsule, granule, powder, pill, solution, emulsion, injection solution, injection, ointment, cream, spray, inhalant, soft gel, liquid, lotion, implant, or transdermal patch.
